(19) United States
(12) Patent Application Publication
(10) Pub. No.: US 2023/0210615 A1
(43) Pub. Date: Jul. 6, 2023

(54) CAMERA CALIBRATION WITH A PATTERN AND CAMERA MOUNTED ON A ROBOTIC MANIPULATOR

(71) Applicant: Asensus Surgical US, Inc., Durham, NC (US)

(72) Inventors: Tal Nir, Haifa (IL); Lior Alpert, Haifa (IL)

(21) Appl. No.: 18/092,288

(22) Filed: Dec. 31, 2022

Related U.S. Application Data
(60) Provisional application No. 63/295,807, filed on Dec. 31, 2021.

(51) Int. Cl. A61B 34/30; A61B 34/00

(52) U.S. Cl. CPC A61B 34/30; A61B 34/70

(57) ABSTRACT

A method of calibrating a camera used for robotic surgery makes use of a target pattern positioned on a robotic manipulator. The camera to be calibrated is moved by the robotic manipulator to move the camera to a plurality of positions and orientations, ideally in closed loop fashion to maintain the target pattern within the camera's image plane. The camera captures images of the target pattern while in the plurality of positions and orientations. A processor receives the captured images and analyzes them to determine calibration parameters for the camera.

This application claims the benefit of U.S. Provisional Application No. 63/295,807, filed Dec. 31, 2021.

### BACKGROUND

Computer vision can be a useful tool for gaining an understanding of a surgical environment. For example, it can be used to estimate 3D measurements between features within an operative site, such as the measurements between instruments disposed at the surgical site, or measurements of anatomical features within the body cavity. Co-pending and commonly owned U.S. application Ser. No. 17/035,534, entitled “Method and System for Providing Real Time Surgical Site Measurements” describes a system and method that use image processing of the endoscopic view to determine sizing and measurement information for a hernia defect or other area of interest within a surgical site. Co-pending and commonly owned U.S. application Ser. No. 17/099,761, entitled “Method and System for Providing Surgical Site Measurements” describes a system and method that use image processing of images of the endoscopic view to estimate or determine distance measurements between identified measurement points at the treatment site.

The measurements may be straight line point to point measurements, or measurements that follow the 3D topography of the tissue positioned between the measurement points.

Camera calibration is essential for such physical 3D measurements using image data, and for other computer vision features such as image distortion correction, image rectification, etc.

Camera calibration solutions typically involve some unique known patterns (fiducials) presented in front of the camera in different poses. A commonly used technique is similar to that described in Z. Zhang, “A flexible new technique for camera calibration,” in IEEE Transactions on Pattern Analysis and Machine Intelligence, vol. 22, no. 11, pp. 1330-1334, November 2000 (“Zhang”). This type of calibration works well but depending on the context in which the camera is to be used, it can delay use of the camera, occupy personnel, and make it difficult to perform “on the fly” calibrations. A camera calibration procedure typically involves printing a checkerboard grid on a planar surface, or using some other designed fiducials, and moving the camera in front of the pattern, or vice versa. In the operating room, calibrating a laparoscopic camera before surgery is a time consuming task that adds to the burden of the operating room staff before surgery. Typical practices requires a staff member to move a calibration pattern in front of the camera, both translating and rotating the pattern in front of the camera while the camera captures images and the associated processors perform the calibration. It would be advantageous to calibrate the camera without occupying the operating room staff with this time-consuming calibration task prior to commencing the procedure.

This application describes a system and method of calibrating a camera in a manner that can be conducted with a reduced amount of interaction on the part of the surgical staff.

### BRIEF DESCRIPTION OF THE DRAWINGS

FIG. 1 shows an example of a calibration system in accordance with the disclosed embodiments.

FIGS. 2 and 3 illustrate a sequence of calibration steps using the system of FIG. 1.

### DETAILED DESCRIPTION

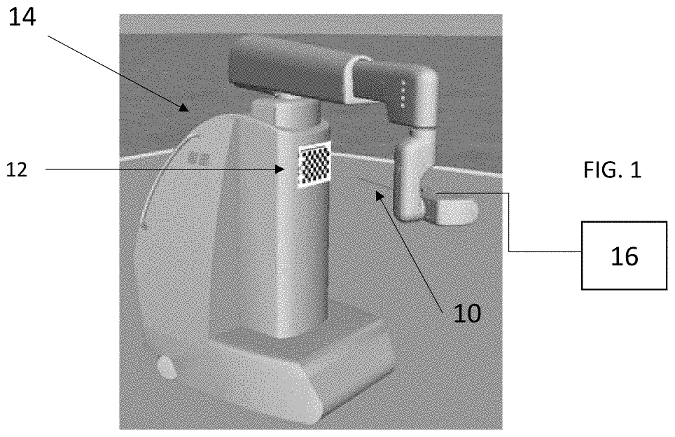

The disclosed calibration is one suitable for use in calibrating cameras that are robotically manipulated during surgery. Components of the system will be described with respect to FIG. 1.

The system includes a camera 10, such as a stereoscopic laparoscopic camera, held by a robotic manipulator arm 14. This is the camera that is to be calibrated in the presently-disclosed calibration exercise. To minimize steps in the procedure, the manipulator arm is ideally the arm that is also used to maneuver the camera during surgery

A target 12 is positioned on a robotic manipulator arm 14. The target is preferably one showing a pattern, possibly a checkerboard pattern or other pattern of the type known to be useful for performing camera calibration sequences. The pattern is shown on a flat surface. It may be printed on the surface, or electronically displayed, such as using a table or other flat display. The squares of the checkerboard pattern are of a known square size. In preferred embodiments, the target 12 is placed on a fixed portion of the manipulator 14.

One or more computers 16 are provided with the system. The computers 16 include a memory storing one or more algorithms executable by the computer to perform one or more tasks. These tasks include receiving the images from the camera, analyzing the images, and detecting the corners of the pattern in the image data. They further include causing movement of the robotic arm in order to displace the corner points to properly cover the image plane. Further tasks may include saving the corner points in a memory associated with the computer for use in the calibration process. Finally, the tasks include estimating the camera parameters based on the detected pattern using an optimization problem solution, a technique that is known to those skilled in the art. See, for example, Z. Zhang, “A flexible new technique for camera calibration,” in IEEE Transactions on Pattern Analysis and Machine Intelligence, vol. 22, no. 11, pp. 1330-1334, November 2000, which is incorporated herein by reference.

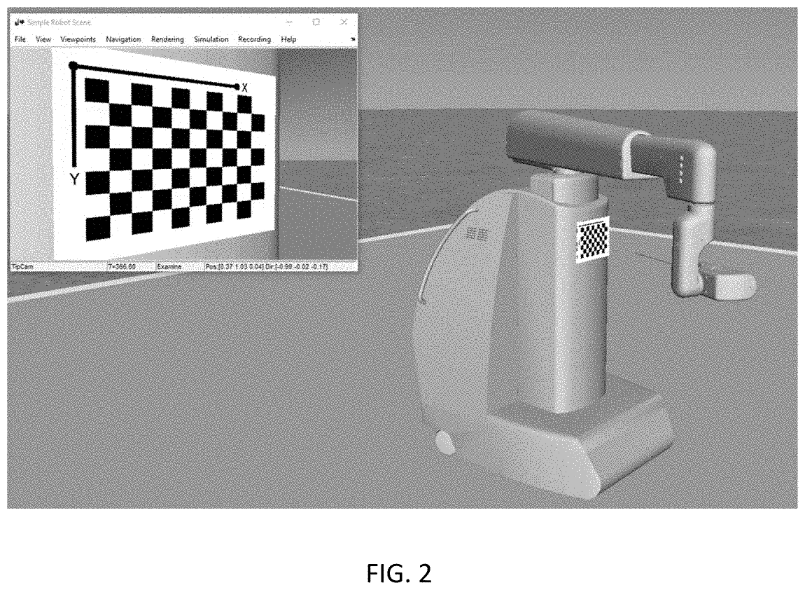

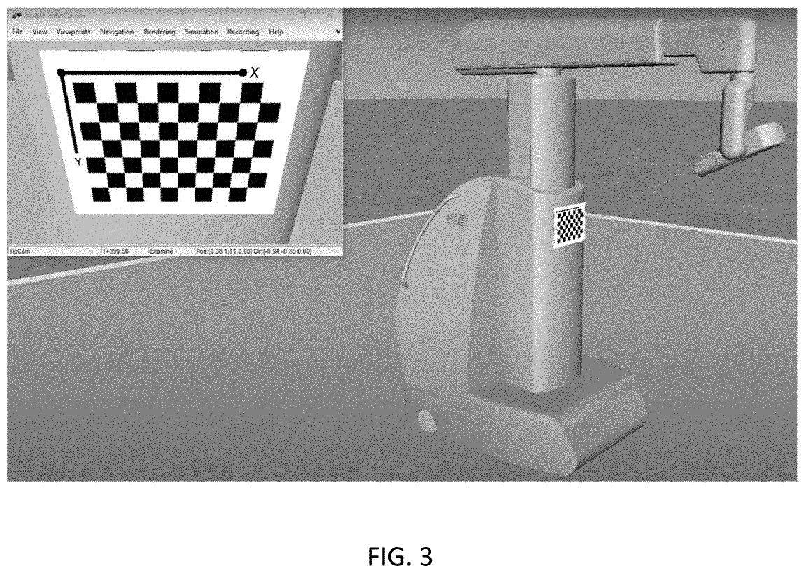

Thus, with a robotically held camera 10, the movement during calibration can be done automatically. The robotic manipulator arm 14 may be moved in a closed loop so that the pattern will move in the captured image, and cover varying locations/sizes/orientations in the image plane. Compare FIG. 2, which shows in the left hand corner an image captured by the camera with the manipulator arm in a first position, with FIG. 3, which shows an image captured by the camera with the manipulator arm in a second, different, position. The pattern is moved to multiple positions and orientations until the calibration parameters are obtained. This method improves the accuracy, reliability, and repeatability of the calibration process results. It also can prevent instances where the pattern is partially outside the field of view of the camera, as such instances result in unusable image frames that may not be used in of the calibration process, resulting in inefficiencies in the process.

For example, it is important for some of the calibration images and points to be close to the image borders in order to accurately estimate the radial distortion parameters. This is easier to achieve consistently when the pattern is moved in the field of view robotically in a closed loop.

The screen target may be permanently affixed to a fixed portion of the manipulator, or a fresh target may be attached prior to a calibration sequence. If the pattern is to be placed on a portion of the manipulator that is covered by a surgical drape, the pattern is sufficiently bold to be readily visible by the camera even when the manipulator is covered by a surgical drape.

In some embodiments, a surgical drape may be provided that has the pattern mounted to or imprinted onto the drape. For those embodiments, the surgical staff will place the drape during pre-operative set up of the system, ensuring that the target is in a fixed location and a planar orientation. To facilitate this, adhesives, fixtures, etc may be used to ensure proper positioning of the target.

With the target in place, the surgical staff can place the system into a camera calibration mode. Once in the calibration mode, images are captured by the camera, with the manipulator repositioning the camera to ensure the capture of images from various positions and orientations. When the system is in the calibration mode, the surgical staff can perform other tasks while awaiting a notification (e.g. an auditory or visual alert) that the calibration sequence is completed. In some cases, ongoing auditory and/or visual alerts may be given by the system during the calibration procedure to ensure staff are aware that the manipulator is moving. The system processors determine the calibration parameters from the captured images using known techniques.

What is claimed is:

1. A method of calibrating a camera used for robotic surgery, comprising the steps of:
(a) positioning a target pattern on a robotic manipulator;
(b) positioning a camera of the robotic manipulator;
(c) causing the robotic manipulator to move the camera to a plurality of positions and orientations;
(d) capturing images of the target pattern using the camera in the plurality of positions and orientations; and
(e) analyzing the captured images to determine calibration parameters for the camera.

2. The method of claim 1, wherein positioning the target pattern on the robotic manipulator includes attaching a planar member with the pattern shown thereon to the robotic manipulator.

3. The method of claim 2, wherein the planar member is a screen displaying the pattern.

4. The method of claim 1, wherein the target pattern on a sterile drape positioned on the robotic manipulator.

5. The method of claim 4, wherein the target pattern is printed or formed on the sterile drape.

6. The method of claim 4, wherein the target pattern is adhered to the sterile drape.

7. The method of claim 1, wherein the robotic manipulator includes a first portion that remains stationary during movement of the camera by the robotic manipulator, and a second portions that moves during movement of the camera by the robotic manipulator, wherein the step of positioning the target pattern comprises attaching the target pattern to the first portion of the robotic manipulator.

8. The method of claim 1, wherein the step of causing the robotic manipulator to move the camera to a plurality of positions and orientations includes moving the robotic manipulator in a closed loop to maintain the pattern within the image plane of the camera.
